(19) United States
(12) Patent Application Publication
(10) Pub. No.: US 2010/0233253 A1
(43) Pub. Date: Sep. 16, 2010

(54) EXTENDED RELEASE GASTRO-RETENTIVE ORAL DRUG DELIVERY SYSTEM FOR VALSARTAN

(75) Inventors: Nikhil Javant Kavimandan, Cedar Knolls, NJ (US); Jay Parthiban Lakshman, Bridgewater, NJ (US); Arnol Singh Matharu, Bedminster, NJ (US); Joerg Ogorka, Steinen (DE); Alan Edward Royce, Saylorsburg, PA (US); Noel Raj Teelucksingh, South Plainfield, NJ (US)

Correspondence Address: NOVARTIS;CORPORATE INTELLECTUAL PROPERTY, ONE HEALTH PLAZA 101/2, EAST HANOVER, NJ, 07936-1080, US

(73) Assignee: Novartis AG, Basel (CH)

(21) Appl. No.: 12/377,949

(22) PCT Filed: Aug. 29, 2007
(86) PCT No.: PCT/US07/77071

§ 371 Date: Feb. 18, 2009

Related U.S. Application Data
(60) Provisional application No. 60/864,869, filed on Nov. 8, 2006.
(60) Provisional application No. 60/824,104, filed on Aug. 31, 2006.

(51) Int. Cl. A61K 9/48; A61K 9/20; A61K 9/14; A61K 31/41; A61P 7/12; A61P 9/00

(52) U.S. Cl. 424/451; 424/464; 424/484; 514/381

(57) ABSTRACT

An extended release gastro-retentive drug delivery system of Valsartan. The drug delivery system contains a release portion containing the Valsartan, a gastro-retentive portion for retaining the drug delivery system in the stomach and an optional secondary portion for delivering a secondary pulse of Valsartan. In another embodiment, there is provided a swellable unfolding membrane comprising Valsartan for sustained administration of Valsartan to the upper GI tract of a patient.

### FIELD OF THE INVENTION

The present invention relates to an extended release gastro-retentive oral drug delivery system. In one embodiment there is provided a pharmaceutical composition, which has a release portion and a gastric-retentive portion, wherein the active agent, Valsartan or its salt, is released into the stomach or gastro-intestinal tract. In another embodiment, there is provided a swellable unfolding membrane comprising Valsartan for sustained administration of Valsartan to the upper GI tract of a patient.

### BACKGROUND OF THE INVENTION

Many drugs have their greatest therapeutic effect when released in the stomach, particularly when the release is prolonged in a continuous, controlled manner. Drugs delivered in this manner have a lower level of side effects and provide their therapeutic effects without the need for repeated dosages, or with a low dosage frequency. Sustained release in the stomach is also useful for therapeutic agents that the stomach does not readily absorb, since sustained release prolongs the contact time of the agent in the stomach or in the upper part of the small intestine, which is where absorption occurs and contact time is limited. Under normal or average conditions, for example, material passes through the small intestine in as little as 1-3 hours.

Many attempts have been made to devise an extended release gastro-retentive drug delivery system where the dosage form is small enough to ingest and then is retained in the gastro-intestinal area for a long enough time for the active agent to be dissolved and eventually absorbed. For example, many swelling and expanding systems have been attempted. There are dosage forms that swell and change their size thereby floating to the surface. These are mostly monolithic devices and are comprised of drug and the swelling agent. Swelling significantly increases the dosage form size, which has been found to influence the transit properties. The stomach discharges its contents including the non-disintegrated solid dosage form, through the pylorus into the intestine. A drawback with these types of systems is that a highly swellable unitary matrix restricts the release of sparingly soluble drugs and various strengths are easily derived. Recently, the retaining of a unitary swellable dosage form in the stomach has been demonstrated in fed state as shown in as shown in U.S. Pat. No. 5,007,790 (“Sustained-Release Oral Drug Dosage Form”), U.S. Pat. No. 5,582,837 (“Alkyl-Substituted Cellulose-Based Sustained-Release Oral Drug Dosage Forms”), U.S. Pat. No. 5,972,389 (“Gastric-Retentive Oral Drug Dosage Forms for the Controlled Release of Sparingly Soluble Drugs and Insoluble Matter”); U.S. Publication 20050013863 A1 (“Dual Drug Dosage Forms with Improved Separation of Drugs”), U.S. Publication 20030147952 A1 (“Manufacture of Oral Dosage Forms Delivering Both Immediate-release and Sustained-release Drugs IR+SR”), U.S. Publication 20030104062 A1 (“Shell-and-Core Dosage Form Approaching Zero-order Drug Release”), U.S. Publication 20030104053 A1 (“Optimal Polymer Mixtures for Gastric Retentive Tablets”), U.S. Publication 20030044466 A1 (“Pharmacological Inducement of the Fed Mode for Enhanced Drug Administration to the Stomach”), U.S. Pat. No. 6,488,962 (“Tablet Shapes to Enhance Gastric Retentive of Swellable Controlled-release Oral Dosage Forms”), U.S. Pat. No. 6,451,808 (“Inhibition of Emetic Effect of Metformin with 5-HT3 Receptor Antagonists”) and U.S. Pat. No. 6,340,475 B2 (“Extending the Duration of Drug Release Within the Stomach During the Fed Mode”). A significant amount of investigations have been made into lipidic content, size of dosage form, expansion volume, fasted and fed states, variability and geometry. See Warrington et al., Br J Clin Pharmacol, Vol. 19, p. 219S (1985); Ichikawa, Watnabe and Miyake, J Pharm Sci, Vol. 80, No. 11, pp. 1062-1066 (1991); Ichikawa et al., J Pharm Sci, Vol. 80, p. 1153 (1991); Oth, Franz, Timmermans and Moes, Pharm Res, Vol. 9, p. 298 (1992); Meyer, Dressman, Fink and Amidon, Gastroenterology, Vol. 89, p. 805 (1985); Khosla, Feely and Davis, Int J Pharm, Vol. 53, p. 107 (1989); Deshpande, Rhodes Shah and Malick, Drug Dev Ind Pharm, Vol. 22, No. 6, pp. 531-539 (1996); and Hwang, Park and Park, Therap Drug Carrier Syst, Vol. 15, No. 3, p. 243 (1998).

Other systems are floating and buoyancing systems wherein the basic principle is to trap gases within sealed encapsulated cores that can float. See Groning and Heun, Drug Dev Ind Pharm, Vol. 10, p. 527 (1984); Atyabi, Sharma, Mohammad and Fell, J Cont Drug Rel, Vol. 42, p. 25 (1996); Atyabi, Sharma, Mohammad and Fell, J Cont Drug Rel, Vol. 42, p. 105 (1996); Ichikawa, Watnabe and Miyake, J Pharm Sci, Vol. 80, p. 1062 (1991). The development of this system is based on hollow cores containing drug and coated with protective membrane. The trapped air in the cores will help the system to float. The second system is made of cores that, in addition to the drug, contain chemical substances, which generate gases when activated. Several attempts with multi-layers have been also made and depend upon the density of the polymers used.

Another system is a bioadhesive system. Intensive studies have been carried out since this approach was first published by Park and Robinson, Int J Pharm, Vol. 19, p. 107 (1984), on a wide range of natural and synthetic polymers for their bioadhesive properties. Although there is a substantial amount of literature is seen, a successful candidate has yet to be found.

Another system is a pharmaceutical gastroretentive drug delivery systems for the controlled release of an active agent in the gastrointestinal tract which comprises: (a) a single- or multi-layered matrix comprising a polymer that does not retain in the stomach more than a conventional dosage form selected from (1) degradable polymers that may be hydrophilic polymers not instantly soluble in gastric fluids, enteric polymers substantially insoluble at pH less than 5.5 and/or hydrophobic polymers and mixtures thereof; (2) non-degradable polymers; and any mixtures of (1) and (2); (b) a continuous or non-continuous membrane comprising at least one polymer having a substantial mechanical strength; and (c) a drug; wherein the matrix when affixed or attached to the membrane prevents evacuation from the stomach of the delivery system for a period of time of from about 3 to about 24 hours. Such a system is disclosed in U.S. Pat. No. 6,685,962.

Several other techniques such as magnetism and ionic resins have been used as gastro-retentive concepts in the literature. A successful candidate is not reported.

Thus, there remains a need for an extended release gastro-retentive drug delivery system for a poorly soluble drug, such as Valsartan, which has an easy to formulate release portion comprising Valsartan and a gastro-retentive portion such that upon the swelling of the gastro-retentive portion the release of Valsartan is not restricted.

### SUMMARY OF THE INVENTION

An extended release gastro-retentive oral drug delivery system, or pharmaceutical composition, is disclosed herein. The drug delivery system comprises Valsartan and has both a release portion and a gastro-retentive portion. In one embodiment, the release portion comprises swellable or swellable erodable or inert material to allow erosional or diffusional release of Valsartan. The gastro-retentive portion comprises swellable material such that the size of the matrix is greater than 1 cm upon hydration. Alternatively, this portion may be made with inert material such as waxes in which case the size of the device will exceed 1 cm prior to hydration and throughout the dissolution/gastric retention time. Valsartan is present only in the release portion. The swelling of the gastro-retentive portion results in an increase in total volume of less than 50% of the volume of the drug delivery system.

Another aspect of the present invention is related to a process for making the extended release oral drug delivery system of the presentation invention comprising blending Valsartan and a controlled release ingredient; mixing the obtained blend with a lubricant (e.g., magnesium stearate) to form the release portion; mixing excipients and a lubricant (e.g., magnesium stearate) in a blender to obtain a blend to form the gastro-retentive layer; and compressing the layers into bilayer tablets. In another embodiment of this aspect of the invention, the formulation is granulated by dry blending.

Another aspect of the present invention is related to the drug delivery system of the present invention further comprising a secondary portion for delivering a secondary or delayed pulse of Valsartan.

In another embodiment of the extended release gastro-retentive oral drug delivery system there is provided a foldable membrane comprising valsartan. Preferably the foldable membrane comprising valsartan is embedded in a capsule, which upon ingestion and exposure to the aqueous environment the foldable membrane fully opens releasing the drug but maintaining the system in the upper GI tract.

In yet another aspect of the present invention there is provided a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering the drug delivery system of the present invention to a subject in need of such treatment.

In another aspect of the present invention there is provided a use of the drug delivery system of the present invention for the manufacture of a medicament for the treatment and/or prevention of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure.

### BRIEF DESCRIPTION OF THE DRAWINGS

FIG. 1: Various configurations for separated functionalities of dissolution and gastric-retention. (2-a) and (2-b) represent embodiments of the present invention. (2-c) and (2-d) represent configurations which can provide gastric-retentive and secondary release simultaneously Legend: 1. Release portion 2. Gastric-retentive portion and 3. secondary portion for a secondary pulse.

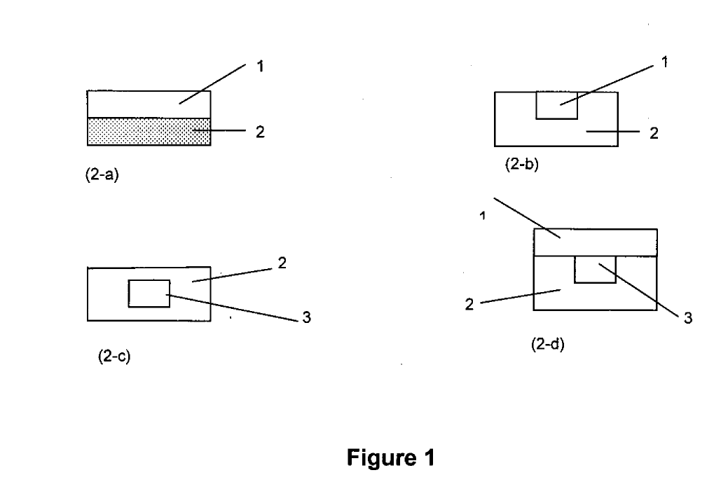

### DETAILED DESCRIPTION OF THE INVENTION

Before describing the present invention in detail, it is to be understood that this invention is not limited to specific active agents, dosage forms, dosing regimens, or the like, as such may vary. It is also to be understood that the terminology used herein is for the purpose of describing particular embodiments only, and is not intended to be limiting.

In describing and claiming the present invention, the following terminology will be used in accordance with the definitions set out below.

The term “dosage form” denotes any form of a pharmaceutical composition that contains an amount of Valsartan sufficient to achieve a therapeutic effect with a single administration.

When the formulation is a tablet or capsule, the dosage form is usually one such tablet or capsule.

The frequency of administration that will provide the most effective results in an efficient manner without overdosing will vary with: (1) the characteristics of the swellable matrix, such as its permeability; and (2) the relative amounts of Valsartan and polymer. In most cases, the dosage form will be such that effective results will be achieved with administration no more frequently than once every eight hours or more, preferably once every 12 hours or more, and even more preferably once every 24 hours or more.

The terms “treating” and “treatment”, as used herein, refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

By an “effective” amount or a “therapeutically effective amount” of a drug or pharmacologically active agent is meant a nontoxic but sufficient amount of the drug or agent to provide the desired effect.

By “pharmaceutically acceptable”, such as in the recitation of a “pharmaceutically acceptable carrier”, or a “pharmaceutically acceptable acid addition salt”, is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative, refers to a derivative having the same type of pharmacological activity as the parent compound and approximately equivalent in degree. When the term, “pharmaceutically acceptable” is used to refer to a derivative (e.g., a salt) of Valsartan, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA.

The term “biocompatible” is used interchangeably with the term “pharmaceutically acceptable”.

The term “soluble”, as used herein, refers to a drug having a solubility (measured in water at 20° C.) in the range of 2% to greater than 50% by weight, more preferably 10% to greater than 40% by weight. The terms “sparingly soluble” and “slightly soluble” refer to a drug having a solubility (measured in water at 20° C.) in the range of 0.001% to about 5% by weight, more preferably 0.001-3% by weight. Such drugs are also referred to as having “low” or “poor” aqueous solubility.

The term “controlled release” is intended to refer to any drug-containing formulation in which release of the drug is not immediate, i.e., with a “controlled release” formulation, oral administration does not result in immediate release of the drug into an absorption pool. The term is used interchangeably with “non-immediate release” as defined in Remington. The Science and Practice of Pharmacy, 19^{th} Ed., Easton, Pa., Mack Publishing Company (1995). As discussed therein, immediate and non-immediate release can be defined kinetically by reference to the following equation:

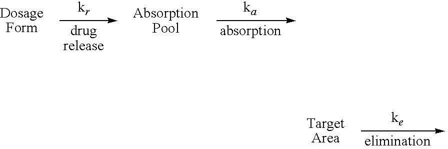

The “absorption pool” represents a solution of the drug administered at a particular absorption site, and kᵣ, kₐ and kₑ₋ are first-order rate constants for: (1) release of the drug from the formulation, (2) absorption and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area. It should be noted that this simplified model uses a single first order rate constant for release and absorption, and that the controlled release kinetics with any particular dosage form may be much for complicated. In general, however, the term “controlled release”, as used herein, includes any non-immediate release formulation including, but not limited to, sustained release, delayed release and secondary release formulations.

The term “sustained release” is used in its conventional sense to refer to a drug formulation that provides for gradual release of a drug over an extended period of time, and that preferably, although not necessarily, results in substantially constant blood levels of a drug over an extended time period.

The term “secondary or delayed pulse” is used in its conventional sense, as the release of a fraction of drug after the most of the extended release portion described above has been released. In effect, this secondary pulse will not be restricted to the stomach area for its complete release and will be in succession to the gastric release.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer”, as used herein, refers to a molecule containing a plurality of covalently attached monomer units, and includes branched, dendrimeric and star polymers, as well as linear polymers. The term also includes both homopolymers and copolymers, e.g., random copolymers, block copolymers and graft copolymers, as well as uncrosslinked polymers and slightly to moderately to substantially crosslinked polymers.

The terms “swellable” and “bioerodible” (or simply “erodible”) are used to refer to the preferred polymers herein, with “swellable” polymers being those that are capable of absorbing water and physically swelling as a result, with the extent to which a polymer can swell being determined by the degree of crosslinking, and “bioerodible” or “erodible” polymers referring to polymers that slowly dissolve and/or gradually hydrolyze in an aqueous fluid, and/or that physically erodes as a result of movement within the stomach or gastrointestinal tract.

The term “fed mode”, as used herein, refers to a state which is typically induced in a patient by the presence of food in the stomach, the food giving rise to two signals, one that is said to stem from stomach distension and the other a chemical signal based on food in the stomach. It has been determined that once the fed mode has been induced, larger particles are retained in the stomach for a longer period of time than smaller particles. Thus, the fed mode is typically induced in a patient by the presence of food in the stomach.

In the normal digestive process, the passage of matter through the stomach is delayed by a physiological condition that is variously referred to as the digestive mode, the postprandial mode, or the “fed mode”. Between fed modes, the stomach is in the interdigestive or “fasting” mode.

The difference between the two modes lies in the pattern of gastroduodenal motor activity.

In the fasting mode, the stomach exhibits a cyclic activity called the interdigestive migrating motor complex (“IMMC”). This activity occurs in four phases:
Phase I, which lasts 45-60 minutes, is the most quiescent, with the stomach experiencing few or no contractions;
Phase II characterized by sweeping contractions occurring in an irregular intermittent pattern and gradually increasing in magnitude;
Phase III consisting of intense bursts of peristaltic waves in both the stomach and the small bowel, lasting for about 5 to 15 minutes; and
Phase IV is a transition period of decreasing activity which lasts until the next cycle begins.

The total cycle time for all four phases is approximately 90 minutes. The greatest activity occurs in Phase III, when powerful peristaltic waves sweep the swallowed saliva, gastric secretions, food particles, and particulate debris, out of the stomach and into the small intestine and colon. Phase III thus serves as an intestinal housekeeper, preparing the upper tract for the next meal and preventing bacterial overgrowth.

The fed mode is initiated by nutritive materials entering the stomach upon the ingestion of food. Initiation is accompanied by a rapid and profound change in the motor pattern of the upper gastrointestinal tract, over a period of 30 seconds to one minute. The change is observed almost simultaneously at all sites along the gastrointestinal tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4-6 hours.

In an aspect of the present invention there is provided an extended release gastro-retentive oral drug delivery system, or pharmaceutical composition comprising a release portion comprising Valsartan and a gastro-retentive portion. In one embodiment, the release portion comprises swellable or swellable erodable or inert material to allow erosional or diffusional release of Valsartan. The gastro-retentive portion comprises swellable material such that the size of the matrix is greater than 1 cm upon hydration. Alternatively, this portion may be made with inert material such as waxes in which case the size of the device will exceed 1 cm prior to hydration and throughout the release/gastric retention period. The swelling of the gastro-retentive portion results in an increase in total volume of less than 50% of the volume of the drug delivery system preferably the increase is less than 40%, more preferably less than 30%, even more preferably less than 20%, more preferably less than 10%, and most preferably less than 5%.

Valsartan or ((S)-N-valeryl-N-{[2′-(1H-tetrazole-5-yl)-biphenyl-4-yl)-methyl}-valine) suitable for use in the present invention can be purchased from commercial sources or can be prepared according to known methods. For example, the preparation of Valsartan is described in U.S. Pat. No. 5,399,578, the entire disclosure of which is incorporated by reference herein. Valsartan may be used for purposes of this invention in its free form as well as in any suitable salt form.

Also included within the scope of the present invention are the salts, esters, amides, prodrugs, active metabolites, analogs, and the like of Valsartan, particularly the calcium salt of Valsartan. A detailed description of the calcium salt and process of making are disclosed in published U.S. Patent Application No. 2003/0207930, the contents of which are fully incorporated by reference herein in their entirety.

The drug delivery system of the present invention is used to administer Valsartan. The transit time through the gastrointestinal tract often limits the amount of drug available for absorption at its most efficient absorption site, or for local activity at one segment of the gastrointestinal tract. The latter is particularly true when the absorption site, or site of local action, is high in the gastrointestinal tract, for example, when the required treatment is local in the stomach as is often the case with ulcers. As the solubility of the drug decreases, the time required for drug dissolution and absorption through the intestinal membrane becomes less adequate and, thus, the transit time becomes a significant factor that interferes with effective drug delivery. To counter this, oral administration of sparingly soluble drugs is done frequently, often several times per day. Moreover, due to their insolubility, sparingly soluble or almost insoluble drugs cannot readily be delivered by either solution-diffusion or membrane-controlled delivery systems. The present dosage forms provide for effective delivery of Valsartan.

In a further embodiment of this aspect of the invention, the dosage form is a bilayer tablet having a gastro-retentive portion comprised of a swellable polymer that erodes over a period longer than the drug delivery period, and a release portion containing Valsartan and being erodible over a drug release period that is predicted using a dissolution test as will be discussed infra. The function of the gastro-retentive portion is to provide sufficient particle size throughout the entire period of drug delivery to enable gastric retention in the fed and fasted mode.

With the present dosage forms, the rate at which Valsartan is released to the gastrointestinal tract is largely dependent on the rate at which the release portion erodes. The polymer used in the release portion of the present invention should not release the drug at too rapid a rate so as to result in a drug overdose or rapid passage into and through the gastrointestinal tract (i.e., in less than about four hours), nor should the polymer release too slowly to achieve the desired biological effect. Thus, polymers that permit a rate of drug release that achieves the requisite pharmacokinetics for a desired duration, as determined using the dissolution test, are selected for use in the dosage forms of the present invention.

The release portion comprises the active ingredient Valsartan and may be comprised of swellable or swellable erodible or inert material. These materials include but are not limited to hydrogels and inert matrices as set forth below

The gastro-retentive portion on the other hand is primarily comprised of swellable material such that the size of the drug delivery system is greater than 1 cm after swelling. The gastro-retentive portion may also be made of an inert material such as wax in such case the drug delivery system will exceed 1 cm prior to hydration. In a preferred embodiment, the gastro-retentive portion swells upon contact with fluids, such as gastric fluid, which results in an increase in total volume of less than 50% of the volume of the system, i.e., tablet, preferably the increase is less than 40%, more preferably less than 30%, even more preferably less than 20%, more preferably less than 10%, and most preferably less than 5%. The 1 cm minimum size is the size necessary to produce gastric retention by not allowing the drug delivery system to pass through the pylorus of the stomach.

Materials suitable for inclusion in the release portion and the gastro-retentive portion of the drug delivery system of the present invention include, but are not limited to, hydrogels, hydrophilic polymers, super disintegrants, gums and inert matrices Water soluble polymer such as methylcellulose, hydroxypropyl cellulose, hydroxypropyl methylcellulose, polyethylene glycol, polyvinyl pyrrolidone. Hydroxypropyl methylcellulose is the preferred polymer in this invention. Certain substitution types, such as 65SH are most preferred.

The inert matrices are made up of those ingredients that do not alter their size and shape when exposed to a dissolution environment. Examples of inert matrices are waxes (fatty acid alcohols such as cetyl alcohol, bees wax and carnauba wax) and insoluble pharmaceutically acceptable polymers, such as, for example, water insoluble cellulosic derivatives, polyvinyl chloride, amino alkyl methacrylates and the like. Suitable water insoluble cellulose derivatives include polymers, such as, for example, ethyl cellulose having a viscosity grade 7 cps, ethyl cellulose having a viscosity grade 10 cps, ethyl cellulose having a viscosity grade 20 cps, ethyl cellulose having a viscosity grade 100 cps.

Hydrophilic and/or hydrophobic materials, such as gums; alkylcelluloses; cellulose ethers, including hydroxyalkylcelluloses and carboxyalkylcelluloses; acrylic resins, including all of the acrylic polymers and copolymers discussed above, and protein derived materials. This list is not meant to be exclusive, and any pharmaceutically acceptable hydrophobic material or hydrophilic material which is capable of imparting the desired sustained release profile of the drug is meant to be included herein. The dosage form may comprise, e.g., from about 1% to about 80% by weight of such material.

Material used in polymer matrices which have been used to achieve controlled release of the drug over a prolonged period of time. Such sustained or controlled release is achieved either by limiting the rate by which the surrounding gastric fluid can diffuse through the matrix and reach the drug, dissolve the drug and diffuse out again with the dissolved drug, or by using a matrix that slowly erodes, continuously exposing fresh drug to the surrounding fluid. Disclosures of polymer matrices that function by either of these two methods are found in U.S. Pat. No. 6,210,710 (“Sustained Release Polymer Blend for Pharmaceutical Applications”, Skinner, inventor, Apr. 3, 2001); U.S. Pat. No. 6,217,903 (“Sustained Release Polymer Blend for Pharmaceutical Applications”, Skinner, inventor, Apr. 17, 2001); International (PCT) Patent Application WO 97/18814 (“Pharmaceutical Formulations”, MacRae et al., inventors, May 29, 1997); U.S. Pat. No. 5,451,409 (“Sustained Release Matrix System Using Hydroxyethyl Cellulose and Hydroxypropyl Cellulose Polymer Blends”, Rencher at al., inventors, Sep. 19, 1995); U.S. Pat. No. 5,945,125 (“Controlled Release Tablet”, Kim, inventor, Aug. 31, 1999); International (PCT) Patent Application WO 96/26718 (“Controlled Release Tablet”, Kim, inventor, Sep. 6, 1996); U.S. Pat. No. 4,915,952 (“Composition Comprising Drug, HPC, HPMC, and PEO”, Ayer et al., inventors, Apr. 10, 1990); U.S. Pat. No. 5,328,942 (“Seed Film Compositions”, Akhtar et al., inventors, Jul. 12, 1994); U.S. Pat. No. 5,783,212 (“Controlled Release Drug Delivery System”, Fassihi et al., inventors, Jul. 21, 1998); U.S. Pat. No. 6,120,803 (“Prolonged Release Active Agent Dosage Form for Gastric Retention”, Wong et al., inventors, Sep. 19, 2000); and U.S. Pat. No. 6,090,411 (“Monolithic Tablet for Controlled Drug Release”, Pillay et al., inventors, Jul. 18, 2000).

The water-swellable polymer forming the matrix in accordance with this invention is any polymer that is non-toxic, that swells in a dimensionally unrestricted manner upon imbibation of water, and that provides for sustained release of an incorporated drug. Examples of polymers suitable for use in this invention are cellulose polymers and their derivatives (such as, for example, hydroxyethylcellulose, hydroxypropylcellulose, carboxymethylcellulose, and microcrystalline cellulose, polysaccharides and their derivatives, polyalkylene oxides, polyethylene glycols, chitosan, poly(vinyl alcohol), xanthan gum, maleic anhydride copolymers, poly(vinyl pyrrolidone), starch and starch-based polymers, poly (2-ethyl-2-oxazoline), poly(ethyleneimine), polyurethane hydrogels, and crosslinked polyacrylic acids and their derivatives. Further examples are copolymers of the polymers listed in the preceding sentence, including block copolymers and grafted polymers. Specific examples of copolymers are PLURONIC® and TECTONIC®, which are polyethylene oxide-polypropylene oxide block copolymers available from BASF Corporation, Chemicals Div., Wyandotte, Mich., USA.

The terms “cellulose” and “cellulosic”, are used herein, to denote a linear polymer of anhydroglucose. Preferred cellulosic polymers are alkyl-substituted cellulosic polymers that ultimately dissolve in a predictably delayed manner. Preferred alkyl-substituted cellulose derivatives are those substituted with alkyl groups of 1-3 carbon atoms each. Examples are methylcellulose, hydroxymethyl-cellulose, hydroxyethylcellulose, hydroxypropylcellulose, hydroxypropylmethylcellulose, and carboxymethylcellulose. In terms of their viscosities, one class of preferred alkyl-substituted celluloses includes those whose viscosity is within the range of about 3 to about 110,000 centipoise as a 2% aqueous solution at 20° C. Another class includes those whose viscosity is within the range of about 1,000 to about 4,000 centipoise as a 1% aqueous solution at 20° C. Particularly preferred alkyl-substituted celluloses are hydroxyethylcellulose and hydroxypropylmethylcellulose. A presently preferred hydroxyethylcellulose is NATRASOL® 250HX NF (National Formulary), available from Aqualon Company, Wilmington, Del., USA.

Polyalkylene oxides of greatest utility in this invention are those having the properties described above for alkyl-substituted cellulose polymers. A particularly preferred polyalkylene oxide is poly(ethylene oxide), which term is used herein to denote a linear polymer of unsubstituted ethylene oxide. Low viscosity poly(ethylene oxide) polymers are preferred. These are products of Union Carbide Chemicals and Plastics Company Inc. of Danbury, Conn., USA.

Polysaccharide gums, both natural and modified (semi-synthetic) can be used. Examples are dextran, xanthan gum, gellan gum, welan gum and rhamsan gum.

Cross-linked polyacrylic acids of greatest utility are those whose properties are the same as those described above for alkyl-substituted cellulose and polyalkylene oxide polymers.

Three presently preferred examples are CARBOPOL® NF grades 971 P, 974P and 934P (BFGoodrich Co., Specialty Polymers and Chemicals Div., Cleveland, Ohio, USA). Further examples are polymers known as WATER LOCK®, which are starch/acrylates/acrylamide copolymers available from Grain Processing Corporation, Muscatine, Iowa, USA.

The water-swellable polymers can be used individually or in combination. Certain combinations will often provide a more controlled release of the drug than their components when used individually. Examples are cellulose-based polymers combined with gums, such as hydroxyethyl cellulose or hydroxypropyl cellulose combined with xanthan gum. Another example is poly(ethylene oxide) combined with xanthan gum.

Release modifiers: pH modifying agents, such as acidic or alkaline substances; and water imbibing agents, such as sodium chloride; or water repelling agents (hydrophobic substances, such as waxes) can be used as release modifiers individually or in combination.

Drugs delivered from the gastric-retentive, controlled delivery dosage forms of the present invention continuously bathe the stomach and upper part of the small intestine, in particular, the duodenum, for many hours. This is considered the “absorption window” for Valsartan. This “absorption window”, particularly the upper region of the small intestine, is the site of most efficient absorption. By continually supplying Valsartan to its most efficient site of absorption, the dosage forms of the present invention allow for more effective oral use of Valsartan.

Since the dosage forms of the present invention provide Valsartan by means of a continuous delivery instead of the pulse-entry delivery associated with conventional dosage forms, two particularly significant benefits result from their use: (1) a reduction in side effects, for example, gastrointestinal irritation; and (2) an ability to effect treatment with less frequent administration of Valsartan. Further, the dosage forms of the present invention, reduce the number of daily doses to one with a lower incidence of side effects.

Other active agents may also be administered in combination using the drug delivery system of the present invention. Examples of particularly important drug combination products include, but are not limited to, combination with metformin hydrochloride, vancomycin hydrochloride, captopril, enalopril or its salts, erythromycin lactobionate, ranitidine hydrochloride, sertraline hydrochloride, ticlopidine hydrochloride, amoxicillin, cefuroxime axetil, cefaclor, clindamycin, doxifluridine, gabapentin, tramadol, fluoxetine hydrochloride, ciprofloxacin hydrochloride, acyclovir, levodopa, ganciclovir, bupropion, lisinopril and a diuretic. Specific examples of diuretics include triampterine, furosemide, bumetanide and hydrochlorothiazide. Alternatively, either of these diuretics can advantageously be used in combination with a beta-adrenergic blocking agent such as propranolol, timolol or metoprolol. These particular combinations are useful in cardiovascular medicine, and provide advantages of reduced cost over separate administrations of the different drugs, plus the particular advantage of reduced side effects and enhanced patient compliance.

The benefits of this invention will be achieved over a wide range of drug loadings, with the weight ratio of drug to polymer generally, although not necessarily, ranging from 1:1000 to about 85:15, typically from 1:500 to about 85:15, more typically from 1:400 to about 80:20.

Preferred loadings (expressed in terms of the weight percent of drug relative to total of drug and polymer) are those within the range of approximately 10-80%, more preferably within the range of approximately 30-80%, and most preferably, in certain cases, within the range of approximately 30-70%. For some applications, however, the benefits will be obtained with drug loadings as low as 0.01%, as may be inferred from the aforementioned ratios.

Another aspect of the present invention is related to the drug delivery system of the present invention further comprising a secondary portion for delivering a secondary pulse of Valsartan.

The dissolution is performed in USP apparatus II (rotation speed of 50 rpm) using a suitable buffer.

Another aspect of the present invention is related to a process for making the extended release oral drug delivery system of the present invention comprising blending Valsartan and a controlled release ingredient; mixing the obtained blend with a lubricant (e.g., magnesium stearate) to form the release portion; mixing excipients and a lubricant (e.g., magnesium stearate) in a blender to obtain a blend to form the gastro-retentive portion; and compressing the layers into bilayer tablets. In another embodiment of this aspect of the invention, the formulation is granulated by dry blending.

The formulations of this invention are typically in the form of tablets. Tablets and capsules represent the most convenient oral dosage forms, in which cases solid pharmaceutical carriers are employed.

Tablets may be manufactured using standard tablet processing procedures and equipment.

One method for forming tablets is by direct compression of a particulate composition, with the individual particles of the composition comprised of a release portion having Valsartan incorporated therein and a gastro-retentive portion, optionally one or more carriers, additives, or the like are also included. As an alternative to direct compression, tablets can be prepared using wet-granulation or dry-granulation processes. Tablets may also be molded rather than compressed, starting with a moist or otherwise tractable material, and using injection or compression molding techniques using suitable molds fitted to a compression unit. Tablets may also be prepared by extrusion in the form of a paste, into a mold, or to provide an extrudate to be “cut” into tablets. However, compression and granulation techniques are preferred, with direct compression particularly preferred.

Tablets prepared for oral administration according to the invention, and manufactured using direct compression, will generally contain other inactive additives such as binders, lubricants, disintegrants, fillers, stabilizers, surfactants, coloring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose and the like) and Veegum. Lubricants are used to facilitate tablet manufacture, promoting powder flow and preventing particle capping (i.e., particle breakage) when pressure is relieved. Useful lubricants are magnesium stearate (in a concentration of from 0.25-3 wt. %, preferably 0.5-1.0 wt. %), calcium stearate, stearic acid, and hydrogenated vegetable oil (preferably comprised of hydrogenated and refined triglycerides of stearic and palmitic acids at about 1-5 wt. %, most preferably less than about 2 wt. %).

Disintegrants are used to facilitate disintegration of the tablet, thereby increasing the erosion rate relative to the dissolution rate, and are generally starches, clays, celluloses, algins, gums, or crosslinked polymers (e.g., cross-linked polyvinyl pyrrolidone). Fillers include, for example, materials such as silicon dioxide, titanium dioxide, alumina, talc, kaolin, powdered cellulose, and microcrystalline cellulose, as well as soluble materials, such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

As noted above, the active agent/polymer matrix particles of the invention may also be administered in packed capsules. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

The bilayer tablet is composed of a gastro-retentive portion that is primarily swellable and a release portion that is primarily erodible, wherein the swellable gastro-retentive portion is composed of at least one primarily swellable polymer, and the erodible release portion is composed of primarily an erodible polymer. Valsartan is present only in the release portion.

Preferred swellable gastro-retentive portion in the bilayer tablets of the invention are hydrogels, e.g., hydroxypropyl methylcelluse and polyalkylene oxides. Optimal high molecular weight poly(ethylene oxide) have number average molecular weights of at least 4 million, preferably at least 5 million, and most preferably 7 million or more. One example of a suitable poly(ethylene oxide) having a number average molecular weight on the order of 7 million is Polyox 303 (Union Carbide). The swellable polymer will generally represent at least 90 wt. %, preferably at least 95 wt. %, and most preferably at least 99 wt. % of the swellable layer, with the remainder of the swellable layer composed of one or more inactive additives as described in Section V. In an exemplary embodiment, the swellable layer contains a lubricant such as magnesium stearate (in a concentration of from 0.25-3 wt. %, preferably from about 0.5-1.0 wt. %), calcium stearate, stearic acid, or hydrogenated vegetable oil (preferably comprised of hydrogenated and refined triglycerides of stearic and palmitic acids at about 1-5 wt. %, most preferably less than about 2 wt. %).

The preferred lubricants are magnesium and calcium stearate.

The erodible release portion in the bilayer tablets is preferably composed of one or more lower molecular weight polyalkylene oxides as well as other hydrophilic polymers, including crosslinked hydrophilic polymers. Preferred lower molecular weight polyalkylene oxides have number average molecular weights in the range of about 200,000 to 2,000,000, and exemplary such polymers that are available commercially include Polyox WSRN-60K, Polyox WSR 1105 and Polyox WSR N-80, having number average molecular weights of 2 million, 900,000 and 200,000, respectively. Other preferred components of the erodible layer of the bilayer tablet are as follows: additional hydrophilic polymers, such as poly (N-vinyl lactams), particularly poly(vinylpyrrolidone) (PVP) (e.g., Povidone); disintegrants, such as cross-linked polymers, e.g., cross-linked poly(vinylpyrrolidone), for example, Crospovidone; fillers, such as microcrystalline cellulose, lactose, lactose monohydrate, and lubricants, such as magnesium stearate and others.

The erodible release portion may comprise, for instance: about 30 wt. % to about 55 wt. %, preferably about 35 wt. % to about 45 wt. % polyalkylene oxide; about 0.25 wt. % to about 3 wt. % magnesium stearate; about 2.5 wt. % to about 20 wt. % disintegrant; and about 5 wt. % to about 35 wt. % filler. In exemplary bilayer tablets of the invention, Valsartan will represent approximately 5-15 wt. % of the release portion.

As with the other types of dosage forms described herein, the bilayer tablets will generally provide for release of at least 80%, preferably at least 85%, and most preferably at least 90%, of the active agent over a time period in the range of about 2-8 hours. In addition, in this embodiment, the in vivo disintegration time of the release portion should be at least two hours shorter than the in vivo disintegration time of the gastro-retentive portion.

Another embodiment of the present invention relates to a pharmaceutical gastroretentive drug delivery system for the controlled release of an active agent in the gastrointestinal tract, which system comprises: a) a single- or multi-layered matrix having a two- or three-dimensional geometric configuration comprising a polymer that does not retain in the stomach more than a conventional dosage form, said polymer selected from: (1) a degradable polymer selected from: i) a hydrophilic polymer which is not instantly soluble in gastric fluids; ii) an enteric polymer substantially insoluble at pH less than 5.5; iii) a hydrophobic polymer; and iv) any mixture of at least two polymers as defined in (i), (ii) or (iii); (2) a non-degradable; and (3) a mixture of at least one polymer as defined in (1) with at least one polymer as defined in (2); b) a continuous or non-continuous membrane, that does not retain in the stomach more than a conventional dosage form, affixed or attached to said matrix, said membrane comprising at least one polymer having a substantial mechanical strength; and c) Valsartan contained within a drug-containing means being embedded in a layer of said matrix, or being entrapped between at least two layers of said matrix, or being attached to said delivery system, wherein said matrix when affixed or attached to said membrane prevents evacuation from the stomach of said delivery system for a period of time of from about 3 to about 24 hours, preferably from about 8 to about 12 hours.

In order to control the mechanical strength, erosion and release characteristics of the Valsartan contained in the delivery device, pharmaceutically acceptable, non-toxic fillers may optionally be added to the matrix, membrane or shielding layer. Examples of such fillers are starch, glucose, lactose, inorganic salts such as sodium or potassium chloride, carbonates, bicarbonates, sulfates, nitrates, silicates and alkali metals phosphates and oxides.

The membrane should control the gastroretentivity of the system by maintaining the system in its desired configuration for predetermined time. Evacuation of the system from the stomach should take place after the shielding layer (or if it does not exist then the matrix layer) undergoes biodegradation, bioerosion, dissolution or disintegration, thus enabling separation of the membrane to its smaller fragments or collapse of the membrane and inevitably the system in any other way.

The membranes used in the device of the invention have substantial mechanical strength. Such membranes may comprise, for example, cellulose ethers and other cellulose derivatives such as cellulose nitrate, cellulose acetate, cellulose acetate butyrate or cellulose acetate propionate; polyesters, such as polyethylene terephthalate, polystyrene, including copolymers and blends of the same; polylactides, including copolymers thereof with p-dioxanone, polyglycolides, polylactidglycolides; polyolefins, including polyethylene, and polypropylene; fluoroplastics, such as polyvinylidene fluoride and polytetrafluoroethylene, including copolymers of the same with hexafluoropropylene or ethylene; polyvinylchloride, polyvinylidene chloride copolymers, ethylene vinyl alcohol copolymers, polyvinyl alcohols, ammonium-methacrylate copolymers and other polyacrylates and polymethacrylates; polyacrylonitriles; polyurethanes; polyphthalamides; polyamides; polyimides; polyamide-imides; polysulfones; polyether sulfones; polyethylene sulfides; polybutadiene; polymethyl pentene; polyphenylene oxide (which may be modified); polyetherimides; polyhydroxyalkanoates; tyrosine derived polyarylates and polycarbonates including polyester carbonates, polyanhydrides, polyphenylene ethers, polyalkenamers, acetal polymers, polyallyls, phenolic polymers, polymelamine formaldehydes, epoxy polymers, polyketones, polyvinyl acetates and polyvinyl carbazoles.

In one preferred example, the membrane comprises a mixture of 1-poly(lactic acid) (1-PLA) and ethylcellulose, at a ratio of 9:1, respectively.

The membrane may contain, or be replaced by a suitable inert metal, e.g. titanium, or inert metal alloys, incorporated into the delivery system of the invention. Such metals or metal alloys serve in preventing the device from rapidly diminishing upon administration.

In a preferred embodiment, the gastroretentive delivery device of the invention may further comprise a shielding layer covering at least one face of said matrix and optionally covering all or part of said membrane. The shielding layer comprises a polymer that does not retain in the stomach more than a conventional dosage form, selected from the group consisting of (a) a hydrophilic polymer which is not instantly soluble in gastric fluids; (b) an enteric polymer substantially insoluble at pH less than 5.5; (c) a hydrophobic polymer; and (d) any mixture of at least two polymers as defined in any of (a), (b) or (c).

The delivery system of the invention comprises a pharmaceutically effective amount of Valsartan, which may optionally be contained in a drug-containing means. Valsartan may be in the form of raw powder, or soluted, dispersed or embedded in a suitable liquid, semisolid, micro- or nanoparticles, micro- or nanospheres, tablet, capsule or a suitable matrix. Valsartan may be embedded in at least one layer of the matrix of the delivery system of the invention. Alternatively, in an embodiment having a multi-layered matrix, preferably a bi-layered matrix, Valsartan may be entrapped between any two of said layers, whether in free form or contained within a drug-containing means. For example, Valsartan in the semi-solid state may be contained between any two layers of the matrix. Another example is that in which Valsartan is contained in a tablet, a capsule or any pharmaceutically compatible matrix, and the drug-containing tablet, capsule or pharmaceutically compatible matrix are entrapped between any two layers of the matrix. Such multi-layered embodiments preferably have a shielding layer. Alternatively, Valsartan, preferably contained with said drug-containing means, may be tethered by tethering means, or otherwise attached, to the delivery system of the invention.

It may be advantageous to further coat the device of the invention with an anti-adhering material, which can be affixed to outer surface/s of the device. Such a material may be any inert, non-swelling material which will prevent self-adhesion of the outer layers (e.g. the matrix or shielding layer) of the device upon hydration thereof. The anti-adhering material may be, for example, cellulose or a cellulose derivative, a silicate, such as magnesium silicate or aluminum silicate, or an enteric polymer substantially insoluble at pH less than 5.5. One preferred example for such a material, used as the anti-adhering layer, is microcrystalline cellulose.

To facilitate administration, the delivery device of the present invention may be administered in a folded configuration. The device is preferably folded into a capsule, preferably a gelatin capsule. In such embodiments it is preferred that the device be further coated with the said pharmaceutically acceptable anti-adhering layer, to prevent its outer layers from adhering to each other when in folded configuration, thus enabling it to unfold during the wetting process in the gastric lumen after administration thereof.

Additionally, folded devices may further comprise a gas-forming agent, not intended for inflation or buoyancy of the device, but rather for providing internal pressure, allowing the folded device to unfold after administration of the capsule and its dissolution in the stomach. The gas-forming agent may be a liquid gas-forming agent which boils at body temperature (34.degree. C.-40.degree. C.), or a solid gas-forming agent. An example for a solid agent is any suitable carbonate, such as calcium carbonate, sodium carbonate or sodium hydrogen carbonate, with sodium hydrogen carbonate being preferred. Liquid gas-forming agents may be methyl formate, tetramethyl silane, iso-pentane, isomers of perfluoropentane, diethyl or diethenyl ether. The gas-forming agent may be in combination with said matrix, or directly or indirectly affixed thereto

The delivery device of the invention may further optionally comprise a pharmaceutically acceptable plasticizer. The plasticizer may be contained in any of the parts of the device, for example in the matrix, in the shielding layer or in the membrane. The plasticizer may be any suitable plasticizing agent, as known to the man of the art. For example, the plasticizer may be an ester, such as a phthalate ester, phosphate ester, citrate ester, fatty acid ester and tartarate ester, glycerine or glycol derivatives, or sorbitol. A preferred plasticizer to be contained in the shielding layer is glycerine.

Each of the components of the device may be affixed to other components, to form the device, by any conventional method known to the man of the art of pharmacy and drug design, for example, by heating or melting each layer, or by using compatible conventional adhesive materials, such as .alpha.-cyanoacrylates, acrylic or methacrylic adhesives, epoxides or plasticized polyvinyl adhesives. However, ‘gluing’ of the layers is preferably performed with organic solvents, which slightly dissolve the polymers, such as ethyl alcohol, acetone, methylene chloride, chloroform or carbon tetrachloride.

The hydrophilic polymer suitable for the various components of the delivery system of the invention may be any hydrophilic polymer which, following suitable treatment if necessary, is not instantly soluble in gastric fluids, such as a protein, a polysaccharide, a polyacrylate, a hydrogel or any derivative of these polymers.

Examples of proteins are proteins derived from connective tissues, such as gelatin and collagen, or an albumin such as serum albumin, milk albumin or soy albumin. In preferred embodiments, the hydrophilic polymer is gelatin or a gelatin derivative, preferably enzymatically hydrolyzed gelatin. A specific example is enzymatically hydrolyzed gelatin having a molecular weight of 10,000-12,000.

Examples of suitable polysaccharides are sodium alginate or carboxymethylcellulose.

Other hydrophilic polymers may be polyvinyl alcohol, polyvinyl pyrrolidone or polyacrylates, such as polyhydroxyethylmethacrylate.

The hydrophilic polymer may be cross-linked with a suitable cross-linking agent. Such cross-linking agents are well known to the man of the art of pharmacy and drug design. These may be, for example, aldehydes (e.g. formaldehyde and glutaraldehyde), alcohols, di-, tri- or tetravalent ions (e.g. aluminum, chromium, titanium or zirconium ions), acyl chlorides (e.g. sebacoyl chloride, tetraphthaloyl chloride) or any other suitable cross-linking agent, such as urea, bis-diazobenzidine, phenol-2,4-disulfonyl chloride, 1,5-difluoro-2,4-dinitrobenzene, 3,6-bis-(mercuromethyl)-dioxane urea, dimethyl adipimidate, N,N′-ethylene-bis-(iodoacetamide) or N-acetyl homocysteine thiolactone. Other suitable hydrogels and their suitable cross-linking agents are listed, for example, in the Handbook of Biodegradable Polymers [A. J. Domb, J. Kost 8, D. M. Weisman, Eds. (1997) Harwood Academic Publishers], incorporated herein by reference. A preferred cross-linking agent is glutaraldehyde.

The enteric polymer is a polymer that is substantially insoluble in a pH of less than 5.5. Such polymers, generally called enteric polymers, are used in the pharmaceutical industry for enteric coating of tables. Examples are shellac, cellulose acetate phthalate, hydroxypropyl methylcellulose phthalate, hydroxypropyl methylcellulose acetate succinate or methylmethacrylate-methacrylic acid copolymers.

There are several advantages in including an enteric polymer in the matrix or the shielding layer, as enteric polymers have improved mechanical properties (e.g. Young's modulus and yield strength). The addition of an enteric polymer to the shielding layer prevented rapid rupture of the shielding layer in vitro A further advantage of using an enteric polymer is to ensure the complete dissolution and/or disintegration of the components of the device, e.g. the matrix, the shielding layer or the membrane, in the intestine, had it not already occurred in the stomach. A preferred enteric polymer incorporated into the shielding layer may be methylmethacrylate-methacrylic acid copolymer, at a ratio of 2:1 ester to free carboxylic groups.

According to a specific embodiment of the invention, the matrix comprises Valsartan embedded in an enteric polymer. In one such specific embodiment, which comprises the matrix, membrane and a shielding layer, the shielding layer comprises about 50% of the hydrophilic polymer which has been suitably cross-linked to reduce its solubility, about 30% enteric polymer and about 20% plasticizer, preferably glycerine.

The matrix or the shielding layer of the delivery device may contain solely or in combination a degradable or non-degradable hydrophobic polymer.

Examples of non-degradable hydrophobic polymers which may be employed within the delivery device of the invention are ethylcellulose or an acrylic acid-methacrylic acid esters copolymer, having from about 5 to 10% functional quaternary ammonium groups. Other suitable polymers are polyethylene, polyamide, polyvinylchloride, polyvinyl acetate and mixtures thereof. Since such non-degradable polymers do not undergo erosion/degradation, when they are employed in the matrix, the size of the matrix or its mechanical properties should not prevent the device from leaving the stomach.

Examples of degradable hydrophobic polymers are poly(.alpha.-hydroxyacids), for example, poly(lactic acid), poly(glycolic acid), copolymers and mixtures of the same.

In embodiments in which Valsartan is contained in the delivery device, rather than being tethered or attached thereto, the role of the matrix is to contain Valsartan. In cases where the polymer/polymer mixtures constructing the matrix are not instantly soluble in gastric fluid, the shielding layer is optional, whereas in cases where Valsartan is embedded in a liquid solution or suspension, in any kind of semisolid such as a gel, ointment or cream or in an instantly soluble polymer film or matrix, which are, in turn, embedded within a layer of the matrix or entrapped between layers of the matrix, the shielding layer becomes essential.

The role of the shielding layer is to maintain the physical integrity of the delivery system (in other words to help in the attachment of the matrix and the membrane), as well as assist in controlling the release rate of Valsartan from the delivery system. The shielding layer should not by itself control the gastroretentivity of the system, except indirectly, by assisting in the attachment of the matrix to the membrane.

Evidently, all the components of the delivery system of the invention are inert, pharmaceutically compatible substances. By “inert” is meant not reacting with the active drug or affecting its properties in any other manner, or itself producing a biological or other effect, particularly adverse effect, upon administration to the treated subject. By “pharmaceutically compatible” is meant not producing a biological or other effect particularly adverse effect upon administration to the treated subject.

### Dosage and Administration

The dose of Valsartan is specified in terms of drug concentration and administration frequency. In contrast, because the dosage forms of the present invention deliver Valsartan by continuous, controlled release, a dose of medication used in the disclosed systems is specified by drug release rate and by duration of release. The continuous, controlled delivery feature of the system allows for: (a) a reduction in drug side effects, since only the level needed is provided to the patient; and (b) a reduction in the number of doses per day. Valsartan is employed in an amount typically ranging from about 40 mg to about 640 mg, preferably from about 40 mg to about 320 mg, and more preferably from about 80 mg to about 320 mg. The amount of Valsartan noted above refers to the amount of free Valsartan present in a dosage form.

In yet another aspect of the present invention there is provided a method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a therapeutically effective amount of the drug delivery system of the present invention to a subject in need of such treatment.

In another aspect of the present invention there is provided a use of the drug delivery system of the present invention for the manufacture of a medicament for the treatment and/or prevention of hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure.

The following examples are illustrative of the invention without limiting it.

### EXAMPLE

The method described below was employed to obtain a bi-layer drug delivery system, the composition of which is set forth in Tables 1 and 2.

TABLE 1
Layer 1

| | | % |
|---|---|---|
| # | Ingredient | Var 102 |
| 1 | Valsartan Calcium DS | 66 |
| 2 | Avicel 102 | 18 |
| 3 | Methocel K100LVP | 8 |
| 4 | NaCl | 6 |
| 5 | Mag. Stearate | 2 |
| | | 100 |

TABLE 2
Layer 2

| # | Ingredient | % |
|---|---|---|
| 1 | Avicel 102 | 37.8 |
| 2 | Methocel E4M | 40 |
| 3 | Methocel K100LVP | 20 |
| 5 | Yellow Iron oxide | 0.2 |
| 6 | Mag. Stearate | 2 |
| | | 100 |

Appropriate weights of Valsartan or its salt, suitable adjuvant, release modifiers and release controlling components (weights shown in Tables 1 and 2) are intimately mixed for use in preparing sustained release portion (Layer 1) and gastroretentive layer (Layer 2), both in combination as a bi-layer tablet, complete the total function of the formulations of the present invention.

For layer I, the active agent is first mixed with Avicel, Methocel and Sodium Chloride in a turbula mixer 10 minutes. Finally, magnesium stearate is added to the blender and mixed for another few minutes. For layer II, Avicel, Methocel (one or more grade), coloring agents such as Yellow iron oxide and Sodium Chloride in a turbula mixer 10 minutes. Finally, magnesium stearate is added to the blender and mixed for another few minutes. These two powder blends are then compressed using a suitable bi-layer tablet press using oval tolling.

### Example 2

The method described below was employed to obtain a bi-layer drug delivery system, the composition of which is set forth in Tables 3 and 4.

TABLE 3
Layer 1

| | | % |
|---|---|---|
| # | Ingredient | B12 |
| 1 | Valsartan Calcium DS | 73 |
| 2 | Avicel 102 | 5 |
| 3 | Methocel 65SH-50cps | 10 |
| 4 | NaCl | 10 |
| 5 | Mag. Stearate | 2 |
| | | 100 |

TABLE 4
Layer 2

| # | Ingredient | % |
|---|---|---|
| 1 | Avicel 102 | 37.8 |
| 2 | Methocel E4M | 40 |
| 3 | Methocel K100LVP | 20 |
| 5 | Yellow Iron oxide | 0.2 |
| 6 | Mag. Stearate | 2 |
| | | 100 |

Appropriate weights of Valsartan or its salt, suitable adjuvant, release modifiers and release controlling components (weights shown in Tables 3 and 4) are intimately mixed for use in preparing sustained release portion (Layer 1) and gastroretentive layer (Layer 2), both in combination as a bi-layer table, complete the total function of the formulations of the present invention.

For layer 1, the active agent is first mixed with Avicel, Methocel and Sodium Chloride in a turbula mixer 10 minutes. Finally, magnesium stearate is added to the blender and mixed for another few minutes. For layer 2, Avicel, Methocel (one or more grade), coloring agents such as Yellow iron oxide and Sodium Chloride in a turbula mixer 10 minutes. Finally, magnesium stearate is added to the blender and mixed for another few minutes. These two powder blends are then compressed using a suitable bi-layer tablet press using oval tolling.

### Example 3

The method described below was employed to obtain a bi-layer drug delivery system, the composition of which is set forth in Tables 5 and 6.

TABLE 5
Layer 1

| | | % |
|---|---|---|
| # | Ingredient | Var B17 |
| 1 | Valsartan Calcium DS | 66 |
| 2 | Avicel 102 | 4 |
| 3 | Methocel 65SH-400cps | 8 |
| 4 | NaCl | 20 |
| 5 | Mag. Stearate | 2 |
| | | 100 |

TABLE 6
Layer2

| # | Ingredient | % |
|---|---|---|
| 1 | Avicel 102 | 37.8 |
| 2 | Methocel E4M | 40 |
| 3 | Methocel K100LVP | 20 |
| 5 | Yellow Iron oxide | 0.2 |
| 6 | Mag. Stearate | 2 |
| | | 100 |

Appropriate weights of Valsartan or its salt, suitable adjuvant, release modifiers and release controlling components (weights shown in Tables 5 and 6) are intimately mixed for use in preparing sustained release portion (Layer 1) and gastroretentive layer (Layer 2), both in combination as a bi-layer table, complete the total function of the formulations of the present invention.

For layer 1, the active agent is first mixed with Avicel, Methocel and Sodium Chloride in a turbula mixer 10 minutes. Finally, magnesium stearate is added to the blender and mixed for another few minutes. For layer 2, Avicel, Methocel (one or more grade), coloring agents, such as Yellow Iron oxide and Sodium Chloride in a turbula mixer 10 minutes. Finally, magnesium stearate is added to the blender and mixed for another few minutes. These two powder blends are then compressed using a suitable bi-layer tablet press using oval tolling.

Additional drug delivery systems of the present invention are shown in Table 7 below. These formulations are made using the steps described above.

TABLE 7

| | 102 | B12 | (B16) | B17 | B19 | B20 | (B21) | B22 |
|---|---|---|---|---|---|---|---|---|
| Layer I | | | | | | | | |
| Valsartan Calcium DS | 66 | 62 | 73 | 66 | 70 | 73 | 66 | 72 |
| Avicel 102 | 18 | 4 | 5 | 4 | 4 | | 4 | 5 |
| Metolose 65SH-50 cps | | 12 | 10 | | 10 | 10 | | 10 |
| Metolose 65SH-400 cps | | | | 8 | | | 5 | |
| Methocel K100LV | 8 | | | | | | | |
| NaCl | 6 | 20 | 10 | 20 | 14 | 15 | 23 | 12 |
| Mag. Stearate | 2 | 2 | 2 | 2 | 2 | 2 | 2 | 2 |
| | 100 | 100 | 100 | 100 | 100 | 100 | 100 | 100 |
| Layer I wt | 607 | 646 | 548 | 607 | 576 | 551 | 607 | 560 |
| Layer II | | | | | | | | |
| Avicel 102 | 38 | 38 | 38 | 38 | 38 | 38 | 38 | 38 |
| Methocel E4M | 40 | 40 | 40 | 40 | 40 | 40 | 40 | 40 |
| Methocel K100LVP | 20 | | 20 | 20 | 20 | 20 | 20 | 20 |
| Metolose 65SH-50 CPs | | 20 | | | | | | |
| Yellow Iron oxide | 0.2 | 0.2 | 0.2 | 0.2 | 0.2 | 0.2 | 0.2 | 0.2 |
| Mag. Stearate | 2 | 2 | 2 | 2 | 2 | 2 | 2 | 2 |
| | 100 | 100 | 100 | 100 | 100 | 100 | 100 | 100 |
| Layer II wt | 538 | 538 | 538 | 538 | 538 | 538 | 538 | 538 |

### Example 4

The method described below was employed to obtain a bi-layer drug delivery system, the composition of which is set forth in Table 8.

TABLE 8
Layer I:

| Materials | | | Qnty/Tab |
|---|---|---|---|
| Layer 1: | PN | % | (mg) |
| Valsartan calcium salt | 5000581.001 | 71.54 | 400.96 |
| Avicel PH102 | 103266 | 4.46 | 25.00 |
| Metolose 65SH-50cp | 970188 | 9.82 | 55.04 |
| Sodium Chloride | 103045 | 11.96 | 67.05 |
| Mag Stearate | 100217 | 2.21 | 12.39 |
| Layer 1 Weight: | | 100.00 | 560.45 |

Layer 2:

| | | | Qnty/Tab |
|---|---|---|---|
| Layer 2: | PN | % | (mg) |
| Avicel PH102 | 103266 | 24.55 | 125.1 |
| Methocel 60HG 4000cp | 100166 | 40.00 | 203.8 |
| Methocel K100 LVP | 133985 | 20.00 | 101.9 |
| KlucelHF | 107836 | 15.00 | 76.4 |
| Iron Oxide, yellow | 100247 | 0.20 | 1.0 |
| Calcium Stearate | 100114 | 0.25 | 1.3 |
| Total wt | | 100.00 | 509.6 |

Layer 1 is prepared for compression as follows: The active, controlled release polymer and other excipients, excluding the lubricant are added to a suitable bin and blended until uniformity is achieved. The blend is then screened through a mesh and blended again. The lubricant is added and the mixture is blended for suitable revolutions.

Layer 2 is prepared as followed: The polymer and other excipients, excluding the lubricant are added to a suitable bin and blended until uniformity is achieved. The blend is then screened through a mesh and blended again. The lubricant is added and the mixture is blended for suitable revolutions.

The layer 1 and layer 2 are then compressed into bilayer tablets on a suitable press.

### Example 5

Additional drug delivery systems of the present invention are shown in Table 9 below. This formulation is made using the steps described in Example 4.

TABLE 9
Layer 1:

| Materials | | | Qnty/Tab |
|---|---|---|---|
| Layer 1: | PN | % | (mg) |
| Valsartan calcium salt | 5000581 | 62.0% | 400.96 |
| Avicel PH102 | 103266 | 6.4% | 41.39 |
| Metolose 65SH-50cp | 970188 | 17.0% | 109.94 |
| Sodium Chloride | 103045 | 12.5% | 80.84 |
| Mag Stearate | 100217 | 2.1% | 13.58 |
| Layer 1 Weight: | | 100.0% | 646.7 |

Layer 2:

| | | | Qnty/Tab |
|---|---|---|---|
| Layer 2: | PN | % | (mg) |
| Avicel PH102 | 103266 | 24.55 | 106.4 |
| Methocel 60HG 4000 | 100166 | 40.00 | 173.3 |
| cp | | | |
| Methocel K100 LVP | 133985 | 20.00 | 86.7 |
| KlucelHF | 107836 | 15.00 | 65.0 |
| Iron Oxide, yellow | 100247 | 0.20 | 0.9 |
| Calcium Stearate | 100114 | 0.25 | 1.1 |
| Total wt | | 100.00 | 433.3 |

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A gastro-retentive drug delivery system for delivering Valsartan or a pharmaceutically acceptable salt thereof to the stomach, duodenum and upper small intestine of a patient comprising:
(a) a release portion comprising Valsartan; and
(b) a swellable gastro-retentive portion,
wherein the swelling of the gastro-retentive portion results in an increase of total volume of the system of less that 50%.

2. The drug delivery system of claim 1, wherein the release portion comprises a hydrogel or inert material.

3. The drug delivery system of claim 1, wherein the gastro-retentive portion is comprised of a material which maintains its size and shape above 1 cm.

4. The drug delivery system of claim 1, wherein Valsartan is present in an amount of between 40 and 320 mg.

5. The drug delivery system of claim 1, wherein Valsartan is the calcium salt of Valsartan.

6. The drug delivery system of claim 1 further comprising an secondary portion for delivering a secondary pulse of Valsartan.

7. A method of treating hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure comprising administering a therapeutically effective amount of a gastro-retentive drug delivery system comprising:
(a) a release portion comprising Valsartan; and
(b) a swellable gastro-retentive portion,
wherein the swelling of the gastro-retentive portion results in an increase of total volume of the system of less that 50% to a subject in need of such treatment.

8. A process for preparing a gastro-retentive drug delivery system comprising the steps of:
(a) blending Valsartan and a controlled release ingredient;
(b) mixing the obtained blend with at least one lubricant to form the release portion;
(c) blending excipients and at least one lubricant to form the gastro-retentive layer; and
(d) compressing the portions into bilayer tablets.

9. The process of claim 8, comprising the additional steps of forming a secondary portion and compressing the secondary portion with the release and gastro-retentive portions to form a bilayer tablet.

10. (canceled)

11. A foldable pharmaceutical gastroretentive drug delivery system for the controlled release of Valsartan in the gastrointestinal tract, which system comprises: a) a single- or multi-layered matrix having a two- or three-dimensional geometric configuration comprising a polymer that does not retain in the stomach more than a conventional dosage form, said polymer selected from the group consisting of: (1) a degradable polymer selected from: (i) a hydrophilic polymer which is not instantly soluble in gastric fluids; (ii) an enteric polymer substantially insoluble at pH less than 5.5; (iii) a hydrophobic polymer; and (iv) any mixture of at least two polymers as defined in (i), (ii) or (iii); (2) a non-degradable polymer; and (3) a mixture of at least one polymer as defined in (1) with at least one polymer as defined in (2); b) a continuous or non-continuous membrane, that does not retain in the stomach more than a conventional dosage form, affixed or attached to said matrix, said membrane comprising at least one polymer having a substantial mechanical strength; and c) Valsartan, which may be in a particulate form or optionally contained within a drug-containing means; Valsartan contained within a drug-containing means being embedded in a layer of said matrix, or being entrapped between at least two layers of said matrix, or being attached to said delivery system, wherein said matrix when affixed or attached to said membrane prevents evacuation from the stomach of said delivery system for a period of time of from about 3 to about 24 hours.

12. The delivery system as claimed in claim 11, further comprising a shielding layer covering at least one face of said matrix, optionally covering all or part of said membrane, said shielding layer comprising a polymer that does not retain in the stomach more than a conventional dosage form, said polymer being selected from the group consisting of: (a) a hydrophilic polymer which is not instantly soluble in gastric fluids; (b) an enteric polymer substantially insoluble at pH less than 5.5; (c) a hydrophobic polymer; and (d) any mixture of at least two polymers as defined in any of (a), (b) or (c).

13. A drug delivery system as claimed in claim 12, wherein Valsartan is in the form selected from the group consisting of a raw powder, a powder soluted, dispersed or embedded in a suitable liquid, semisolid, micro- or nanoparticles, micro- or nanospheres, a tablet, a capsule or a suitable specific two- or three-dimensional matrix.

14. The delivery system as claimed in claim 11, further comprising a suitable plasticizer.
